(19) United States
(12) Patent Application Publication
(10) Pub. No.: US 2025/0366995 A1
(43) Pub. Date: Dec. 4, 2025

(54) MEDICAL DEVICE BASED ON BIOCERAMICS, ITS USE AS A SYNTHETIC BONE GRAFT AND PROCESS FOR THE PREPARATION THEREOF

(71) Applicant: M3 HEALTH INDÚSTRIA E COMÉRCIO DE PRODUTOS MÉDICOS, ODONTOLÓGICOS E CORRELATOS S.A., Jundiaí (BR)

(72) Inventors: Alberto BLAY, São Paulo (BR); Elaine YOSHIKO MATSUBARA, São Paulo (BR); Jamil AWAD SHIBLI, Guarulhos (BR); Leticia JORDÃO MARQUES DE OLIVEIRA, Jundiaí (BR); Luiz Antonio SASKA JUNIOR, Araraquara (BR); Sybele SASKA SPECIAN, Santana de Parnaiba (BR); Samy TUNCHEL; Livia PILATTI MENDES DA SILVA, Jundiaí (BR)

(21) Appl. No.: 18/872,914

(22) PCT Filed: Jun. 12, 2023
(86) PCT No.: PCT/BR2023/050193

§ 371 Date: Dec. 9, 2024

Related U.S. Application Data
(60) Provisional application No. 63/351,030, filed on Jun. 10, 2022.

(51) Int. Cl. A61F 2/28; A61F 2/30; A61L 27/08; A61L 27/12; B28B 1/00; B33Y 50/00; B33Y 80/00

(52) U.S. Cl. CPC A61F 2/2846; A61F 2/30771; A61F 2/30942; A61L 27/08; A61L 27/12; B28B 1/001; B33Y 50/00; B33Y 80/00; A61F 2002/2835; A61F 2002/285; A61F 2002/30199; A61F 2002/3092; A61F 2002/30948; A61F 2002/30952; A61F 2002/30985; A61F 2310/00173; A61F 2310/00293; A61L 2430/02

(57) ABSTRACT

The present invention relates to a medical device manufactured using the additive manufacturing process (3D printing). It is a medical device used preferably as a bone graft composed of a porous structure based on bioceramics based on β-tricalcium phosphate (β-TCP) or hydroxyapatite, which may or not contain nanostructures in its composition, for example: carbon nanostructures (graphene, graphene oxide, reduced graphene oxide, carbon nanotubes, etc.) and, in preferred embodiments, stem cells and polymeric membrane. Also, the present invention relates to the use of this device as a bone graft and the process of preparing this device.

### FIELD OF INVENTION

The present invention relates to a medical device which is manufactured from the additive manufacturing process (3D printing). It is a medical device used as a bone graft composed of a porous structure based on bioceramics, β-tricalcium phosphate (β-TCP) or hydroxyapatite (HA), which may or not contain carbon nanostructures (graphene, graphene oxide, reduced graphene oxide, carbon nanotubes, etc.) in preferred embodiments, it may carry stem cells.

Also, the present invention relates to the use of this device as a bone graft and the process for the preparation of this device.

### BACKGROUND OF THE INVENTION

Filling materials and synthetic bone substitutes made of porous bioceramics have excellent physical, chemical and biological properties, being classified as bioactive and osteoconductive, which means that the osteoblast is able to use the material as a scaffold to initiate bone formation in the region. Bioceramics based on calcium phosphate, Ca₃(PO₄)₂, stand out due to their chemical composition and crystalline structures that are similar to the inorganic matter of the human body, the crystalline phases being hydroxyapatite (HA) and tricalcium phosphate (β-TCP), the most used ones in biological systems, in particulate or block form.

Calcium phosphate blocks have superior mechanical and morphological properties compared to particulate synthetic bone substitutes. The interconnected pores and high porosity facilitate cell migration for bone reconstruction, diffusion of nutrients and can even serve as vehicles for the controlled release of drugs and molecules.

We highlight below some teachings of the prior art that refer to the present matter:

Document U.S. Pat. No. 9,079,357 discloses an improved method for layering a molded body made of highly viscous light-curing material, the possibility of use in the dental industry being mentioned.

Document U.S. Pat. No. 9,403,726 describes a process for the preparation of high strength photopolymerizable ceramic wedges, which can be used as ceramic molded parts or articles, dental inlays, onlays, veneers, crowns, bridges, and structures. The process uses a paste based on a radically polymerizable binder, polymerization initiator and filler, which comprises at least one acidic photoinitiator monomer and ceramic and/or glass-ceramic particles. The aforementioned ceramic and glass-ceramic particles have dental applications and can be used for the preparation of dental restorations such as inlays, onlays, veneers, crowns, bridges, or frameworks.

Document CN 105943406 discloses a 3D printing composite material for mouth rehabilitation and a method of preparing and using the 3D printing composite material. 3D printing composite material for oral rehabilitation is small in healing retraction, short in healing time and high in antibacterial properties, and is suitable for manufacturing dental prosthesis.

The document PI0912499-3 describes the use of tricalcium phosphate and hydroxyapatite biomaterials in surgeries that require a bone substitute such as grafting or other indications.

The document PFAFFINGER, M. et al. “Stabilization of tricalcium phosphate slurries against sedimentation for stereolithographic additive manufacturing and influence on the final mechanical properties.” International Journal of Applied Ceramic Technology, v. 14, no. 4, p. 499-506, 2017 discloses the use of lithography (LCM) for the production of ceramic pieces and highlights the use of a paste filled with tricalcium phosphate.

The document EBRAHIMI M. & BOTELHO M. “Biphasic calcium phosphates (BCP) of hydroxyapatite (HA) and tricalcium phosphate (TCP) as bone substitutes: Importance of physicochemical characterizations in biomaterials studies.” (2017) pages 93-97 discloses the use of tricalcium phosphate as a substitute for use in tissue engineering, mainly in bone regeneration.

The document TAPPA, K., JAMMALAMADAKA, U. “Novel biomaterials used in medical 3D printing techniques”. Journal of Functional Biomaterials, v. 9, no. 1, 2018 describes the ability to build patient-specific implants, including custom implants, using 3D printing technology. It also describes that a wide variety of biomaterials are currently being used in medical 3D printing, including metals, ceramics, polymers and compounds for tissue engineering and regenerative medicine.

Therefore, in the prior art, there is no solution equivalent to the one presented here in the present invention that combines technical differentials, quality, safety, and reliability.

### OBJECTIVES OF THE INVENTION

Thus, it is an objective of the present invention to provide a solution for patients in need of bone graft, mainly in the cranio-maxillofacial region, but other regions of the body that need bone graft are also applicable.

It is another objective of the present invention to provide an application for additive manufacturing in the production of medical devices, constituting the technological differential.

It is another objective of the present invention to offer a technology involving the provision of products and therapeutic approaches, which can be applied to regenerative medicine.

It is another object of the present invention to provide a customized technology produced by additive manufacturing.

It is another object of the present invention to provide a technology that can result in patient-specific medical devices as well as medical devices with predefined shapes and dimensions.

Still, it is another object of the present invention to provide a medical device that may comprise stem cells.

It is another object of the present invention to provide a porous medical device that may promote an improved osteointegration.

It is another object of the present invention to provide a bioceramic medical device comprising carbon nanostructure elements in view to have properties improved.

Further, it is another object of the present invention to provide a medical device being a solution for patients in need of bone graft combining a device made from bioceramic and a membrane made from PDO.

### SUMMARY OF THE INVENTION

The present invention achieves these and other objectives by means of a medical device particularly indicated to be used as a synthetic bone graft, being composed of synthetic bioceramic consisting of B-tricalcium phosphate or hydroxyapatite; being produced by additive manufacturing; and optionally being personalized.

The present invention achieves these and other objectives by means of a process for preparing the above medical device being patient-specific device comprising the following steps:
an image exam of the patient is obtained (example: computed tomography, DICOM file) using CAD software;
a planning is carried out and a Planning Report is generated, which is sent for approval by the client (dentist or physician);
after approval, the file in STL format (binary encoding) is imported by the print preparation software, where parameters related to the raw material are added, and sent to the printer via a wireless or cable connection;
the personalized medical device (custom-made or patient-specific) is produced by additive manufacturing.

The present invention achieves these and other objectives by means of a process for preparing the above medical device being a predefined shape device comprising the following steps:
Technical drawings of standard models (block, wedge and cylinder)
Archives.STL ready and already approved by the project team and technical responsible;
Customize a graft according to the anatomy of the receiving bed with the aid of drills and sterile surgical/prosthetic discs for straight pieces;
Give adequate finish, leaving no sharp edges and corners to avoid perforation of the flap;
Make adaptation of the block to the receiving bed without leaving steps, leveling all sides of the graft to the bone defect.

Furthermore, the present invention achieves these and other objectives by means of a process of inserting the above medical device comprising the following steps:
a. initial site assessment (clinical defect);
b. tissue detachment to expose the surgical bed;
c. prior use of a polymeric guide (bone graft analog) to verify the adaptation of the device of the present invention;
d. perforating the surgical bed to allow blood perfusion and nutrition of said device;
e. adaptation of the medical device above on the surgical bed and stabilization with a screw.

In a second preferred embodiment, the present invention comprises graphene or other compounds made from carbon such as nanocarbon and carbon tubes. The presence of these components adds mechanical strength to the device of the present invention, also conferring antibacterial property.

In a third preferred embodiment, the present invention comprises stem cells preferably being autogenous adult mesenchymal stem cells obtained from dermal punch of the patient.

In a fourth preferred embodiment of the invention, the present invention comprises carbon nanostructures (graphene, graphene oxide, reduced graphene oxide, carbon nanotubes, etc.) and stem cells. Finally, the present invention achieves these and other goals through use of the above device to function as a bone graft both for volume augmentation/reconstruction of craniomaxillofacial or other bone defects and for space maintenance, being gradually replaced by newly formed bone.

In a fifth preferred embodiment of the invention, the present invention is combined to a polydioxanone membrane in view to improve and accelerate guided bone regeneration.

### DETAILED DESCRIPTION OF THE FIGURES

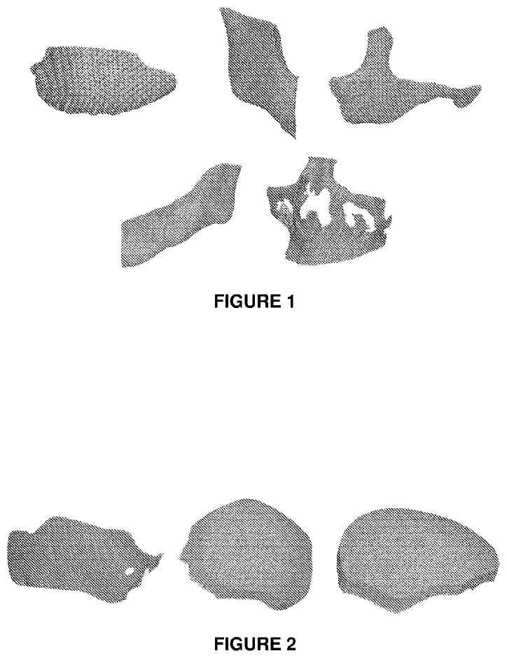

FIG. 1 illustrates examples of the device of the present invention applied to facial bones being personalized (patient-specific medical device);

FIG. 2 illustrates examples of the device of the present invention applied to neurocranium bones being personalized (patient-specific medical device);

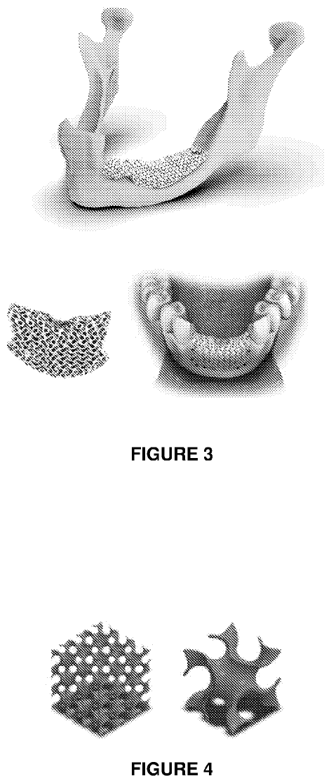

FIG. 3 illustrates an application of the device of the present invention in a jaw being personalized (patient-specific medical device);

FIG. 4 illustrates a cellular solid based on the gyroid surface;

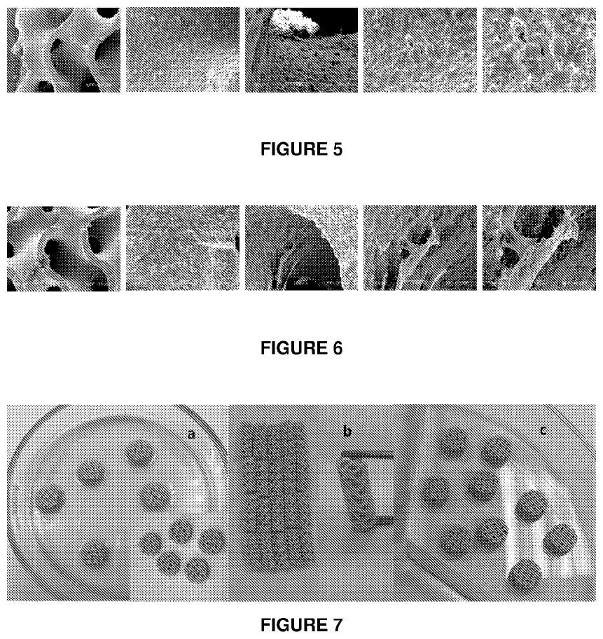

FIG. 5 illustrates in detail the present invention (A) Mesenchymal stem cells—Dermal punch;

FIG. 6 illustrates in detail the present invention (A) Mesenchymal stem cells—Dermal punch and (B) osteogenesis;

FIG. 7 illustrates a preferred embodiment of the present invention: discs; cylinders; discs 5×2 mm;

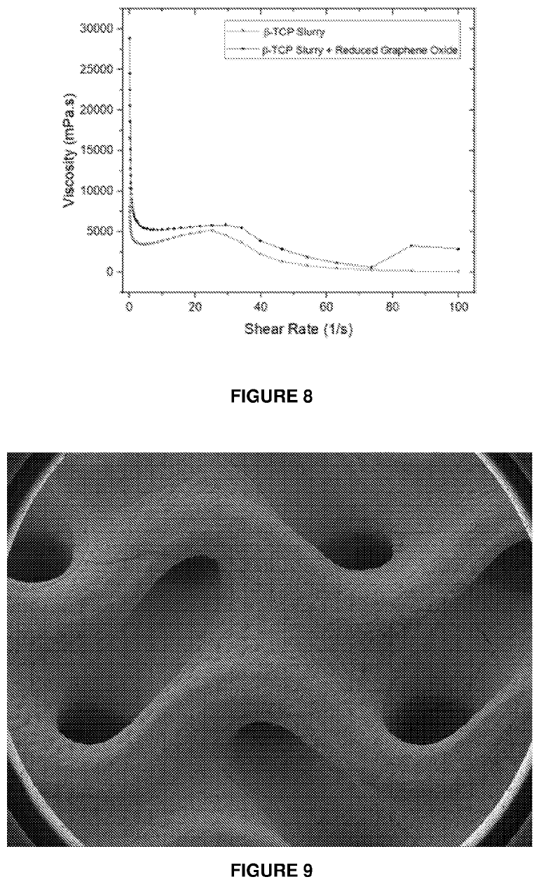

FIG. 8 is a plot which shows properties of the embodiment illustrated in FIG. 7;

FIG. 9 are photomicrographs of a present invention sample at 25× magnification;

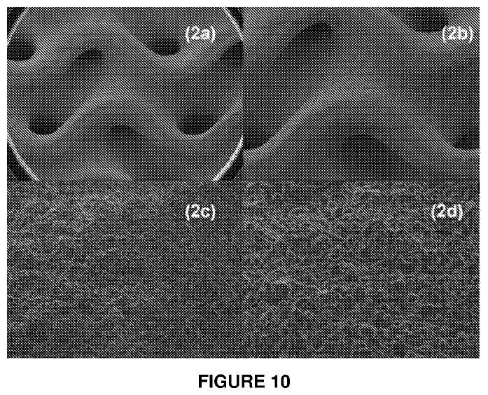

FIG. 10 are photomicrographs of a sample of the present invention in the magnifications of: (2a) 25×; (2b) 37×; (2c) 1.000× and (2d) 2.000×.

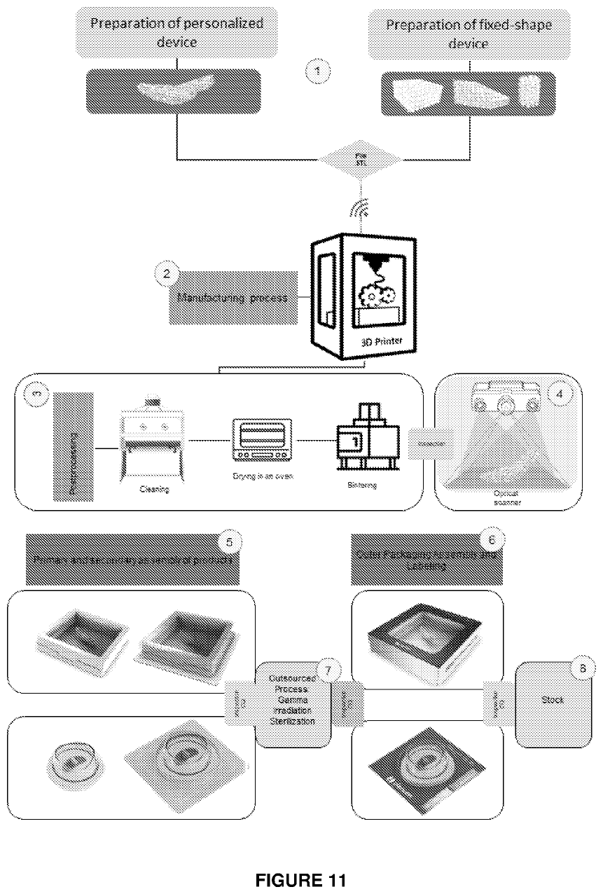

FIG. 11 is a flow of the production process of the present invention;

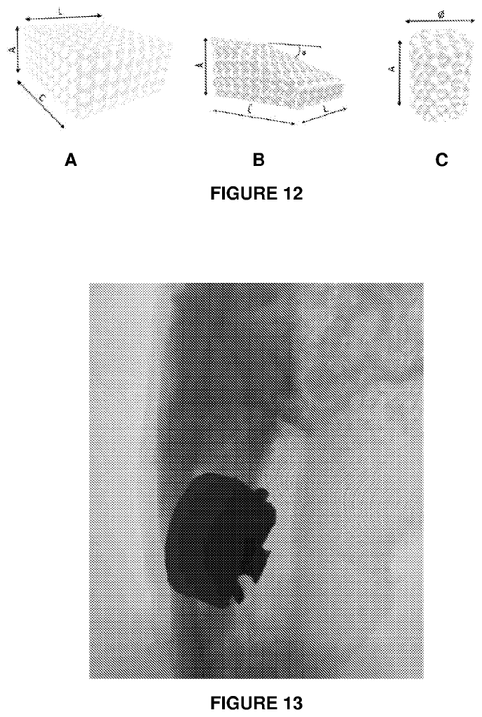

FIG. 12 illustrates shapes of the present invention;

FIG. 13 is a result of an image exam that shows the embodiment of the present invention comprising graphene.

### DETAILED DESCRIPTION OF THE INVENTION

The present invention relates to a medical device used as a synthetic bone graft, preferably composed of synthetic bioceramic consisting of β-tricalcium phosphate (≥95% of β-TCP) or hydroxyapatite (≥95% of HA) being resorbable. This device is intended to be a synthetic bone substitute working as a bone graft both for volume increase/reconstruction of cranio-maxillofacial defects or other bone defects and for space maintenance, being gradually replaced by newly formed bone.

Bioceramics such as calcium phosphate (hydroxyapatite and β-TCP) are materials that induce a controlled reaction with the host tissue in a physiological environment, accelerating the healing process (tissue neoformation). They also favor the cellular mechanisms by colonization of stem cells of the respective tissue to be repaired/regenerated.

Bioceramics promote the proliferation and differentiation of cells to form new tissue, with interaction/binding to the surface of the medical device. Still, they are absorbable.

The present invention aims at osteoconductive and biomimetic properties, so that their structure favors cell recognition and tissue regeneration.

It works as a framework that mimics the trabecular bone microstructure, enabling effective vascularization and bone formation.

Some properties of bioceramics used in the present invention are highlighted below:
increased pH promoted by the release of Ca²⁺ ions;
increased pH stimulates alkaline phosphatase activity in pre-existing osteoblastic cells and in newly differentiated active osteoblasts;
Basic pH induces synthesis of type I collagen and non-collagen proteins plus alkaline phosphatase;
pH at the material-tissue interface is gradually reestablished by enzymatic actions and nucleation of calcium phosphate crystals in collagen fibers (osteoid) until they form a chemically more stable phase (primary bone);
Action of biological buffers containing HCO³⁻ ions favor the precipitation of apatite carbonate.

In this sense, the present invention is a resorbable synthetic bone graft to be used in the reconstruction and/or guided regeneration of bone defects. The macro and microstructure, surface area and the chemical composition confer effective osteoconductivity, high hydrophilic property, controlled resorption/dissolution of the crystalline phase constituent of this bone graft, which is gradually reabsorbed by the body and replaced by neoformed bone tissue during the process of repair or regeneration of bone tissue.

The present invention is a technology that can be applied to result in predefined medical devices as well as in patient-specific medical devices with custom dimensions, manufactured from the additive manufacturing process (3D printing). This medical device is classified as a bone graft, consisting of a porous structure based on bioceramics preferably tricalcium β-phosphate (β-TCP).

In a preferred embodiment of the present invention, the structure of the medical device is the following:
the following structure:

Macropores
Diameter: from 360 μm to 440 μm
Micropores
External surface pores: from 0.62 μm to 0.76 μm
Cross-sectional pores: from 0.55 μm to 0.67 μm
Microstructure
Porosity: from 25.0% to 30.0%%
80% of pores: 0.39 to 5.47 μm
Average diameter: from 0.63 to 0.77 μm
Penetration: from 0.023 cm³/g to 0.029 cm³/g
Average density: from 2.892 g/cm³ to 3.534 g/cm³

The examples of this device illustrated in FIGS. 1 and 2 are classified as patient-specific medical devices, which are designed and built virtually based on data acquired by computed tomography or magnetic resonance imaging using virtual 3D models and CAD/CAM techniques. Subsequently, with this file generated in the planning, the customized product is obtained via additive manufacturing technology.

The virtual/digital planning and design of the device is obtained from a planning through software that treats the images in DICOM format (Digital Imaging and Communications in Medicine) derived from imaging exams (computed tomography), in which the device is designed with complex geometry and faithful to the anatomy of the bone tissue to be reconstructed. In this process of planning the device of the present invention, a STL (Standard Triangle Language) file is obtained, which, associated with additive manufacturing technology, produces these parts with complex geometries that support personalized medical devices, sub-classified as “patient-specific.”

A patient-specific medical device is “a medical device that is made compatible (or that is made compatible) with the anatomy of a patient using scaling techniques based on anatomical references, or using the anatomical features obtained from imaging exams, being typically produced in batches through a process that can be validated and reproduced, under the responsibility of the manufacturer, even if the project can be developed together with the qualified health professional”.

In more detail, the virtual planning of the models of the medical device of the present invention comprises the following steps:
an examination of the patient's images (example: computed tomography, DICOM file) is obtained using CAD software;
a planning is carried out and a Planning Report is generated, which is sent for approval by the client (dental surgeon);
after approval, the file in STL format (in binary encoding) is imported by the print preparation software, where parameters related to raw material are added, and sent to the printer via a wireless or cable connection.

The device of the present invention is indicated for filling and/or reconstructing bone defects in the cranio-maxillofacial region or in other regions. The device of the present invention can also be used for alveolar ridge augmentation/reconstruction or other craniomaxillofacial defects associated with resorbable or non-resorbable membranes, meshes or fabrics for guided tissue regeneration.

The device of the present invention works as a bone substitute, favoring the three-dimensional reconstruction of bone defects or space maintenance, providing defect regeneration. The device of the present invention is slowly reabsorbed by the body, which favors the replacement of the graft by neoformed bone tissue during the tissue repair or regeneration process.

A preferred embodiment of the device of the present invention comprises an internal infill, preferably in the form of a gyroid as illustrated in FIG. 4.

In this embodiment, the filling pattern of the customized blocks is composed of cellular architecture preferably elaborated in CAD, aiming to mimic the bone tissue. The gyroid structure model is a good example of a pattern successfully applied to 3D printing due to high strength combined with lightness.

Regarding the embodiment of the present invention being predefined shapes thereof as illustrated in FIG. 12, some of shapes and dimensions are described below:

Models/Sizes
(A) Length (C) × Width (W) × Height (H)

| | |
|---|---|
| BLOCK-01 | 10 × 10 × 03 mm |
| BLOCK-02 | 10 × 15 × 03 mm |
| BLOCK-03 | 10 × 10 × 05 mm |
| BLOCK -04 | 20 × 20 × 08 mm |
| BLOCK-05 | 20 × 20 × 10 mm |
| BLOCK-06 | 20 × 30 × 08 mm |
| BLOCK-07 | 20 × 30 × 10 mm |

(B) Length (C) × Width (W) × Height (H) - angle

| | |
|---|---|
| WEDGE-01 | 20 × 15 × 6 mm - 9° |
| WEDGE -02 | 20 × 15 × 8 mm - 14° |
| WEDGE -03 | 20 × 15 × 10 mm - 19° |
| WEDGE -04 | 20 × 15 × 12 mm - 24° |
| WEDGE -05 | 20 × 15 × 14 mm - 29° |

(C) Diameter (Ø) × Height (A)

| | |
|---|---|
| CYLINDER-01 | 08 × 10 mm |
| CYLINDER-02 | 10 × 10 mm |
| CYLINDER-03 | 12 × 10 mm |
| CYLINDER-04 | 14 × 10 mm |
| CYLINDER-05 | 08 × 20 mm |
| CYLINDER-06 | 10 × 20 mm |
| CYLINDER-07 | 12 × 20 mm |
| CYLINDER-08 | 14 × 20 mm |

In a preferred embodiment of the present invention, the medical device may comprise carbon nanostructures (graphene, graphene oxide, reduced graphene oxide, carbon nanotubes, etc.). Preferably, the medical device comprises graphene in a concentration that varies from 0.001% to 0.01%.

In a preferred embodiment of the present invention, the medical device may comprise a PDO membrane (polydioxanone membrane). Further, in a preferred embodiment of the present invention, the medical device may comprise a PDO membrane and stem cells. This composition has excellent potential to improve and accelerate guided bone regeneration. Moreover, the use of stem cells in the present invention optimized the bioceramic by increasing its osteoinductive and osteoprogenitor capacity even with the resorption of the present invention.

### Application of the Device of the Present Invention

The present invention is a single-use, invasive, implantable product that will be in contact with bone tissues, soft tissues of the craniomaxillofacial region and with body fluids for a long term (>30 days). Its application can be done on an outpatient basis or in an operating room environment (hospital) by surgeons (dentist or physician) qualified to perform surgical procedures of bone reconstruction/regeneration in the craniomaxillofacial region. The techniques for use and application of the device of the present invention vary according to the preference and technique recommended by the surgeon, and it is up to him to choose the therapeutic approach, approval of the patient-specific medical device previously planned virtually (geometry, dimensions, and positioning of the product to the recipient bed) and approved by the surgeon.

Therefore, a general surgical technique is not recommended for all patients. The surgical protocol must be performed according to the surgeon's references and previous experiences, always considering the most appropriate choice for the bone graft, the technique, the grafting sequence, and the use of membranes, meshes or fabrics, when necessary, always based on conventional and consecrated therapeutic techniques of guided tissue regeneration and bone grafting.

Before use, it is necessary to previously analyze the amount and quality of the soft tissues in the grafting region. Whether the adjacent soft tissues will be sufficient to cover the device of the present invention without tension on the flap, as the choice or planning approved by the client erroneously of said device that will be used, as well as errors in the indication, manipulation, preparation of bone tissue or soft tissues, and installation of the product, can cause damage to the physical structure and contamination of the product and contribute to product failure.

Proper preparation of the site (recipient bed) where the device of the present invention is to be applied is very important. The entire area to be treated must be prepared to favor vascularization of the recipient bed. If necessary, proceed with the decorticalization of the recipient bed or make some perforations with drills up to 2 mm in diameter to promote vascularization and nutrition to the device, with controlled bleeding, so that the bone graft has a perfect adaptation to the recipient bed and achieve the expected results.

### Insertion of the Device of the Present Invention

The insertion of the device of the present invention into the indicated location illustrated in FIG. 3 comprises the following steps:
a. initial site assessment (clinical defect);
b. tissue detachment to expose the surgical bed;
c. prior use of a polymeric guide (bone graft analog, also produced by additive manufacturing) to verify the adaptation of the device of the present invention;
d. perforating the surgical bed to allow blood perfusion and nutrition of said device;
e. adaptation of the device of the present invention on the surgical bed and stabilization with a screw.

It is important to highlight that the device of the present invention must be used by qualified professionals, under sterile surgical field conditions and use of adequate preoperative antisepsis to avoid the risk of contamination of the sterile product.

The device of the present invention is indicated for:
Implantology: bone reconstructions for horizontal and/or vertical augmentation;
Periodontics: filling of intraosseous defects;
Cranio-Maxillo-Facial: traumatology and bone reconstructions for horizontal and/or vertical augmentation (facial bones and neurocranium);
General Clinical: filling of intraosseous bone defects or bone reconstruction of long bones or spine.

The present invention presents numerous technical and economic advantages when compared to the prior art, some of which are listed below:
the device of the present invention is indicated for guided tissue regeneration, augmentation/reconstruction and filling of atrophic bones or intraosseous defects, face bones, neurocranium, spine, long bones or bone defects resulting from congenital, post-traumatic, post-surgical problems, which are not intrinsic to the stability of the bone structure;
the device of the present invention was designed and manufactured ensuring safety and efficacy in relation to toxicity;
the device of the present invention was designed and manufactured ensuring safety and effectiveness in relation to biological compatibility. Physicochemical analyzes and pre-clinical tests ensure its properties for performance for the intended purpose of use;
the device of the present invention is an absorbable product and does not have risks deriving from substances released from them, according to a test for the identification of trace elements and systemic toxicity;
it is a product that has no risk of injury linked to its physical and ergonomic characteristics. It is a patient-specific product, which must adapt perfectly to the bone defect/recipient bed according to each clinical case.
In addition to huge gains in the agility of the surgical process, the product brings a better quality of life for patients, as it rebuilds lost tissues with customized synthetic material, eliminating the need to remove bone tissue from other areas of the patient's body, or the use of grafts from other sources, such as human and animal origin in a non-personalized way. Thus, this product allows precision, predictability and a great reduction of surgical processes, and complications such as infection or rejection of the product in patients.
The three-dimensional macro and microstructures standardized by additive manufacturing of the present invention confer effective osteoconductivity, high hydrophilic propriety, controlled resorption/dissolution of the crystalline phase constituent of this bone graft, which is absorbed by the body gradually and replaced by neoformed bone tissue during the process of repair or regeneration of bone tissue.
The compressive strength property of these bone grafts ensures a mode of use that allows drilling by drills and fixation by means of screws. The adjacent contact of the present invention next to the receptor bed increases the contact area between them, facilitating revascularization and providing the migration of osteogenic cells into the bone graft. In general, resorbable synthetic materials for bone regeneration are the materials of choice; since these materials are free of possible contaminants or organic moles from the homogenous and heterogenous grafts, which may induce immunological responses. In this way, synthetic materials minimize the risk of infection, inflammation and other complications in the postoperative period.
Also, histological analyses of the biocompatibility tests performed for the present invention demonstrated that the design of the projected extrinsic porosity confers on the product the property of osteoconduction and integration into the adjacent bone tissue, as observed in the implantation tests with neoformed healthy bone tissue inside the macropores.
the relevant essential performance requirements for the present invention include:
a) Filling and/or reconstruction of bone defects in the craniomaxillofacial region;
b) Act as a bone substitute, favoring the maintenance of the space of the bone defect providing the repair of the defect or even the increase of bone volume;
c) Be reabsorbed by the body and replaced by neoformed tissue during the process of repair or regeneration of bone tissue.
the relevant essential safety requirements for the present invention include:
(a) chemical composition (raw material);
(b) sterilisation;
c) Mechanical strength;
(d) biological compatibility;
e) Clinical Safety-Adverse Effects.
Clinical investigation has carried out the enlargement of atrophic maxillae;
the present invention acts as a bone substitute, favoring the maintenance of the space of the bone defect providing the repair of the defect or even the increase of bone volume.
the present invention provides maxillary increase and maintenance of this increase/volume.
the present invention is reabsorbed by the body and replaced by newly formed tissue during the process of repair or regeneration of the bone tissue.
further, neoformed tissue was observed inside and adjacent to the present invention after 08 months postoperatively.

### Tests

### A. The Tests Below Referred to the Embodiment of the Present Invention Being the Patient-Specific Medical Device

### Test 01. Mechanical Test

The mechanical test was based on evaluating the compressive strength that the product offers when a force is applied to it. The property of compressive strength is what ensures that the product is capable of being drilled by drills and fixed by means of screws, without unintentional fracture (failure mode). The better the fixation next to the recipient bed, the greater the contact area for vascularization and osteoconduction.

For the composition of β-tricalcium phosphate, the test result identified that the average maximum force reached was 508.42±72.84 N. The average compressive strength calculated was 25.89±3.71 MPa, a value higher than the porous ceramic blocks of similar products already commercialized: ChronOS™ (DepuySynthes) and Adbone (Medbone). For hydroxyapatite, the average maximum force reached was 81.83±23.09N, resulting in 4.17±1.18 MPa of compressive strength.

### Test 02. Chemical/Material Characterization

The raw material used for the production of the device of the present invention is a composite resin based on calcium phosphate bioceramics (in the crystalline phase β-tricalcium phosphate, β-TCP, or hydroxyapatite, HA) and, after the sintering process, only the inorganic phase (bioceramic) was obtained.

The tests carried out to ensure the quality of the raw material used in the manufacturing process, as well as the composition of the final product:

Trace elements: The quantification analysis chosen employs a sensitive and indicated method to determine the limit of specific trace elements, namely arsenic (As), cadmium (Cd), mercury (Hg), lead (Pb) and the sum of metals heavy. In the analyzed samples, both for the composition of β-tricalcium phosphate and for hydroxyapatite, all the quantified analytes presented a result lower than the LQ (Limit of Quantification), concluding that the ceramic prototype in question is safe to be used as a bone graft, regarding residual quantification (toxicity to heavy metals) of trace elements.

Qualitative and quantitative determination of crystalline phases: The mass fractions of the β-tricalcium phosphate phase were quantified by the Rietveld method. The three batches analyzed for the composition attested as β-tricalcium phosphate showed a composition of β-TCP>95%. Additionally, the purity of the crystalline phase was evaluated by means of spectroscopy in the infrared region (FTIR). Meanwhile, the three batches certified hydroxyapatite had a composition of HA >95% and calcium oxide <1%.

Chemical analysis and characterization of crystallinity and phase purity. The FTIR analysis confirmed that the sample analyzed is composed of β-tricalcium phosphate (β-TCP), since the bands identified are consistent with the spectrum in the literature for the compound. It is also concluded that the purity of β-TCP is in accordance with the appropriate since it was proven in the spectrum the absence of characteristic bands of calcium pyrophosphate, both in α and β forms, namely: 434 cm⁻¹, 757 cm⁻¹, 1210 cm⁻¹, 1185 cm⁻¹, 723 cm⁻¹ and 454 cm⁻¹.

Shape and dimension: For both compositions, the analyzed samples presented the appropriate dimensional to the geometry proposed in the project for the specimen submitted to additive manufacturing. In the sintering process of the green pieces, the burning of organic matter and the rearrangement of the ceramic powder particles occurs, forming a strong bond or neck at the contact points of the particles. As the contact increases, the porosity decreases substantially, and the particles approach each other leading to shrinkage of the part. By analyzing the microscopy images of the sintered part, we can therefore estimate the sintering stage of the part, the relative density, and the grain formation profile. The analyzed samples appear to be in the final stage of sintering with normal grain growth, since the pores have closed almost completely, and the grains are well formed and uniform.

Porosity: Although the theoretical density for β-TCP is 3.14 g/cm³, the three batches with β-TCP composition had an average density of 3.61±0.09 g/cm³ and an average surface area of 0.23±0.09 m²/g, disclosing that the sintering process used in the process increases the relative density value. Additionally, pores smaller than 51 μm could not be determined due to the impossibility of mercury penetration at compressions of 0-5000 PSI, demonstrating that the blocks do not have a significant number of intrinsic pores between 51 μm and 0.02 μm. The triplicate of samples printed with hydroxyapatite composition had an average density of 3.3745±0.03037 g/cm³ and an average surface area of 0.1345±0.0820 m²/g. The average porosity determined by mercury intrusion is 11.06±2.58% with an average diameter of 0.56 μm. These data disclose reproducibility and reliability to the manufacturing process at the post-processing stage.

Properties of graft structure such as porosity, interconnectivity, pore size, permeability, and pore shape are widely known to influence osteogenesis in vivo. In the specimens analyzed, the porosity and diameter of the interconnections were estimated by means of mercury intrusion. The total porosity value (micropores) calculated was 28.66%, with penetration of 0.026 (cm³/g), 80% of the interconnected pores with measurements between 5.47-0.39 μm (microporosity) and mean pore diameter of 0.70 μm. In order to calculate the porosity, the actual density (3.213±0.022 g/cm³) was determined by helium gas pycnometry. Additionally, the mean surface area (0.243±0.2 m²/g) was measured using the BET technique (initials of the researchers Brunauer, Emmett and Teller).

The porosity of 28.66% is similar to that reported in other studies that made use of printed β-TCP specimens, using 400 μm pores in the filling architecture. However, modifications in the fill geometry and size of the unit cell can lead to significant variation in porosity. It was concluded, therefore, that the complex geometry of these specimens (gyroid with macropores: theoretical value of Ø 400 μm and real value of 404±0.0238 μm) added to the characteristics of the microstructure determined by these analyses, demonstrate an intrinsic porosity with the presence of interconnected micropores that favor the adsorption of fluids, adhesion and cell proliferation, making this microstructure an excellent factor for a cellular framework.

Dissolution and pH change: For both compositions, the pH of the TRIS-HCl buffer solution did not change by more than 0.2 from the initial value during the assay. The calcium content of the solutions was analyzed by ICP-OES, and the results showed a gradual increase in the concentration of calcium ions during the analyzed period. The mass loss was not significant enough to be quantified (<LQ 0.1%), (LQ, Limit of Quantification).

In this way, these assays guarantee the product's compliance in relation to pH changes when implanted, as a significant change in pH after implant can induce exacerbated inflammatory responses and interfere with the regenerative process and tissue formation. In addition, it is desirable that biomaterials for bone filling/grafting are degraded at a rate close to the formation of new bone tissue. The degradation of a material is primarily governed by its chemical composition and physical characteristics, and the higher the Ca/P molar ratio, the lower the solubility of the material.

### Test 03. Cytotoxicity Assay

The cytotoxicity of a test substance is determined by the percentage of cells that remain viable after exposing a given cell population to a concentration of the test substance extract.

Under the test conditions, the device of the present invention did not promote a considerable reduction in cell viability (>30%) in any of the test groups, showing cell viability of 96%. Therefore, the test item (β-TCP, ≥95%) has no cytotoxic potential. Under the same test conditions, the test item (HA ≥95%) did not reduce cell viability.

### Test 04. Genotoxicity Assay

The micronucleus test detects chromosomal changes during cell division and aims to assess the genotoxic potential of substances. Under the test conditions, the device sample of the present invention showed no genotoxic (mutagenic) effect in the short treatment, with and without metabolic activation and in the continuous treatment. Therefore, under the conditions described, both compositions (hydroxyapatite and β-TCP) were considered non-mutagenic.

### Test 05. Maximized Dermal Sensitization

The Maximized Dermal Sensitization assay consists of analyzing the material's ability to cause an immunologically mediated skin reaction to a substance, characterized by the appearance of edema and erythema. The maximized method uses an adjuvant capable of stimulating the immune response in order to enhance the sensitivity of the method (Freund's Complete Adjuvant—FCA). The LLNA (Local Lymph Node Assay) method assesses the potential for dermal sensitization in rodents.

Under study conditions, test items in both compositions (hydroxyapatite and β-TCP) were classified as non-sensitizing.

### Test 06. Intracutaneous Reactivity

Intracutaneous Reactivity test consists of the evaluation of local adverse effects occurring after the inoculation of a substance intracutaneously in a single dose. Under study conditions, test items in both compositions (hydroxyapatite and β-TCP) did not induce intracutaneous reactivity in rabbits.

### Test 07. Acute Systemic Toxicity

The acute systemic toxicity test is the assessment of a possible health risk and adverse effects caused by a single exposure to a substance. These studies provide information on systemic toxic effects and serve as a basis for estimating the safety of the substance. Under the conditions of the study, the test items in both compositions (hydroxyapatite and β-TCP) meet the requirements of absence of acute systemic toxicity.

### Test 08. Subchronic Toxicity

Subchronic Toxicity test evaluates the possible toxic and systemic effects resulting from the implant of a material in an animal species. In this sense, the present trial aimed to evaluate the possible toxic and systemic effects resulting from exposure to the bone implant of the test item for 90 days in rabbits. Under the conditions of the study, for both compositions (hydroxyapatite and β-TCP) no systemic or toxic signs were identified in the daily clinical evaluations, evolution of body weight, weekly feed consumption, ophthalmological examination and in the biochemical, hematological and anatomopathological measurements related to the subchronic (90 days) systemic exposure to test item implant. In this way, the test item can be classified as non-toxic.

### Test 09. Chronic Toxicity

The Chronic Systemic Toxicity test evaluates the possible toxic and systemic effects resulting from the implant of a material in an animal species. In this sense, the present trial aimed to evaluate the possible toxic and systemic effects resulting from exposure to the bone implant of the test item for 180 days in rabbits. Under the conditions of the study, no systemic or toxic signs were identified in the daily clinical evaluations, evolution of body weight, weekly feed consumption, ophthalmological examination and in the biochemical, hematological and anatomopathological measurements related to chronic systemic exposure (180 days) to the implant of the test. In this way, the test item (both in the composition of hydroxyapatite and β-TCP) can be classified as non-toxic.

### Test 10. Tests in Animal Models

The bone implant assay evaluates the local effects after implant of a material in an animal species. In this sense, the present study aimed to characterize the history and evolution of tissue response after implant of the medical device, evaluating its biological safety and clinical performance similar to the intended purpose of use. Under the conditions of the study, no local or toxic signs were identified in the anatomopathological evaluations regarding chronic systemic exposure (26 weeks) to the implant of the test item. The sum of the results obtained in the present study suggest that the test item (both in the hydroxyapatite and β-TCP composition) has non-irritating characteristics.

### Test 11. Tests of the Embodiment of the Present Invention Comprising Mesenchymal Stem Cells

The integration of MSCs, along with the capacity of osteogenic differentiation, in ceramic blocks was analyzed by electron microscopy. For this, the cells were plated in duplicate, at a density of 106 cells on the scaffolds, where one of the samples was induced for osteogenic differentiation.

The structure of the block showed a strong point of tropism for the cells, where they practically covered their entire structure and there were morphological changes in the sample exposed to the osteogenic differentiation medium, suggesting calcifications points (FIGS. 5 and 6).

### Test 12. Clinical Research Blocks Bone Graft

Title: Use of the present invention in comparison to the autogenous block graft for bone increase in atrophic maxillae thickness. Randomized Split-Mouth Clinical Study

Objective: The aim of this study is to evaluate the safety and efficacy of bone neoformation in the anterior maxillary region, by comparing the use of the present invention and the use of autogenous block grafting.

### Method

All participants underwent cone-beam computed tomography (CBCT) before the surgical procedure;
The files from the computed tomography were sent along with the information of which side of the maxilla the personalized block graft would be used. From the files, the drawings (virtual planning) of the customized blocks of the test group were made in specific software, which were later made by means of additive manufacturing, in a 3D printer suitable for ceramic printing. The customized pieces (present invention) were made using Lithography-based Ceramic Manufacturing (LCM) technology, whose process consists of printing the virtual piece in resin containing the desired bioceramic (slurry), in this case, β-tricalcium phosphate (β-TCP).
Preparation and installation of the autogenous graft;
Installation of the present invention;
Postoperative;
Biopsy: After 8 months of the bone grafting procedure, the participants underwent a new examination.
Analysis: Resorption was calculated by subtracting the results obtained after 8 months from the immediate results. Resorption in relation to the initial volume was used to calculate the resorption rate (% resorption).
Images of CT scans performed were compared shortly after the installation of the grafts (immediate) and after 8 months of repair/regeneration period.

### Results

Bone volumes were evaluated at the time of insertion (immediate postoperative), and eight months after the procedure (Postoperative 8 months), of the autogenous bone and the present invention, of 15 patients. The results are presented in the following table.

Table 1. Results of bone volumes and absorption of autogenous bone and the present invention at the time of insertion (immediate PO), and eight months after the procedure (PO 8 months).

### Descriptive

The results of the statistical analyses are presented in Tables 1 and 2. The results showed that, although there is no difference between immediate volume and volume at 8 months, the resorption rate of the present invention was 8.8 percentage points lower than that of autogenous bone after 8 months. Thus, in terms of volume maintenance, the present invention is superior (p<0.05) than autogenous bone.

TABLE 1
Results of bone volume comparisons using
the ANOVA test with repeated measures.
Results: ANOVA with repeated measures

| | Estimation | | | |
|---|---|---|---|---|
| | of the mean | | | |
| Comparisons* | difference | IC 95% | | P value |
| Immediate (Plenum - | 92.04 | 34.03 | 150.05 | <0.01 |
| Autogenous) | | | | |
| 8 months (Plenum - | 104.56 | 46.55 | 162.57 | <0.01 |
| Autogenous) | | | | |
| Plenum (immediate - | 7.30 | −50.71 | 65.32 | 0.80 |
| 8 months) | | | | |
| Autogenous (immediate - | 19.82 | −38.19 | 77.84 | 0.50 |
| 8 months) | | | | |

*comparisons via orthogonal contrasts

TABLE 2
Resorption results using the paired t-Student test.
Results: Paired t-Student Test

| Comparações entre | Estimativa da | | | |
|---|---|---|---|---|
| grupos (Plenum - | diferença | | | |
| Autogenous) | média | IC 95% | | Value p |
| Resorption (mm³) | −12.52 | −25.93 | 0.89 | 0.07 |
| % Resorption | −8.80 | −15.61 | −1.98 | 0.02 |

Result: In terms of volume maintenance, the present invention demonstrated superiority over autogenous bone.

### Test 13. Macropores—Scanning Electron Microscopy

Specification of diameter of micropores and macropores through measurements from photomicrographs obtained by SEM (Scanning Electron Microscopy), of a section of the material and at the place where the pores are in contact with each other a fictitious limit between the pores must be established.

The technology of the manufacturing process makes it feasible to produce complex porous architectures, so the same filling structure was applied to the specimen used in the present invention, which is the gyroid with a unit cell of 2.4 mm, resulting in macropores with theoretical diameters of 400 μm.

FIG. 9 shows a photomicrograph of the body of evidence performed with magnification of 25×. From the figure, the average diameter of the macropores was determined, using the software ImageJ (Wayne Rasband and contributors, National Institutes of Health, USA).

The calculated mean diameter, 404±0.0238 μm, is consistent with the theoretical diameter of the pores present in the gyroid structure, 400 μm (FIG. 4). The size of the gyroid's macropores was designed for osteoconduction, since results indicate that, to increase bone formation, the pores of the architecture of 3D printed ceramic grafts must be larger than 300 μm with an upper limit of 500 μm.

From the images generated by the Scanning Electron Microscopy (SEM) technique, it was possible to measure the diameter of the macropores present in the complex architecture (with gyroid structure). It is concluded that the specimen (Ø 8 mm×2.3 mm) presented an average macropore diameter of 404±0.0238 μm, a value that corroborates the theoretical diameter projected in CAD, 400 μm.

### Test 14. Micropores-Scanning Electron Microscopy (SEM)

FIG. 10 shows images of the body of evidence in different magnifications.

The micropores were measured on the surface of the body of evidence, with a mean value of 0.69 μm and on the cross-section (inside the specimen), with a mean value of 0.61 μm. The mercury intrusion porosimetry test corroborates the results obtained by SEM, since the value of the average pore diameter is 0.70 μm for the entire piece.

The printed and sintered specimens analyzed presented geometry according to the design (STL) submitted to additive manufacturing. The images showed superficial cracks, which although they do not fragment the piece or cross it entirely, can also be observed internally, in the images of the sectional cut made in the piece. In addition to the analysis of surface and apparent pores, another objective of the assay was the determination of the average diameter of micropores, in which it was possible to measure the pores of the surface and the cross-section (interior) of the specimen, with mean values of 0.69 and 0.61 μm respectively.

### B. The Tests Below Referred to the Embodiment of the Present Invention Being the Predefined-Shape of the Present Invention

### Test 01. Mechanical Characteristics

Tests to ensure the efficacy and safety of the product considering mechanical stresses were carried out. The property of compressive strength is what ensures that the product is capable of drilling by drills and fixation by means of screws, without unintentional fracture (failure mode). The calculated compressive strength, considering the area of the gyroid structure in the cross-section, was 18.83±1.51 MPa, a value of which is higher than the mechanical strength values of comparable ceramic blocks already commercialized.

### Test 02. Chemical Characterization of the Material (Raw Material)

The raw material used for the production of the present invention is a resin based on calcium phosphate bioceramics (in the crystalline phase β-tricalcium phosphate), and after the sintering process only the inorganic phase β-tricalcium phosphate (β-TCP) is obtained. The tests carried out to ensure the quality of the raw material were:
Trace elements: All quantified elements presented results below the stipulated limits.
Qualitative and quantitative determination of crystalline phases: It was concluded that the mass fractions of the quantified β-tricalcium phosphate phase are in accordance with the stipulated limit.
Porosity: It is concluded that the complex geometry, gyroid structure with macropores of Ø 404±0.0238 μm, added to the characteristics of the microstructure determined by these analyses, demonstrate an intrinsic porosity with the presence of interconnected micropores that favor the adsorption of fluids, adhesion and cell proliferation, making this combination of macro and microstructure an excellent factor for a cellular framework.
Dissolution and pH change: The results showed the gradual increase in the concentration of calcium ions during the analyzed period. In this way, these tests guarantee the conformity of the product in relation to the pH change when implanted, since a significant change of pH after implantation can induce exacerbated inflammatory responses and interfere in the regenerative process and tissue formation.
Biological compatibility The results of the characterization tests indicate that the present invention is safe without adverse and harmful effects directly and indirectly related by comparable products in relation to the raw material/chemical composition.
Dissolution and pH change: Within specifications.
Cytotoxicity: Non-cytotoxic product.
Genotoxicity: Non-genotoxic product.
Dermal Sensitization—LLNA: Sensitizing product.
Intracutaneous Reactivity: Non-irritating product.
Acute Systemic Toxicity: Product does not induce acute toxicity.
Bioburden: Average microbial load detected in three different batches of less than 1 CFU/unit, demonstrating a low chance of pyrogenic response due to substances from gram-positive bacteria and fungi.
Bacterial Endotoxins (Clot Gel): Within the limit set for medical devices by the FDA.
Subchronic Systemic Toxicity: Non-toxic product.
Chronic Systemic Toxicity: Non-toxic product.
Implantation (90 days): Non-irritating product.
Implantation (180 days in rabbits): Non-irritating product.

### C. Tests of the Embodiment of the Present Invention Comprising Graphene

Compositions of the present invention comprising graphene in an amount of 0.01% and 0.005% were tested in view to verify its properties as viscosity and shear rate. Some samples were prepared from such composition which are illustrated in FIG. 7. The compositions tested are the following:

| | β-TCP Slurry | β-TCP Slurry | β-TCP Slurry |
|---|---|---|---|
| Composition | rGO 0.01% | rGO 0.005% | rGO 0.005% |
| Multi-Functional | 41.01% | 35.172% | 41.01% |
| Monomer | | | |
| Photoinitiator | 2.1% | 1.840% | 2.1% |
| β-TCP | 57.35% | 61.347% | 57.35% |
| IPA - Isopropyl | 1.53% | 1.636% | 1.53% |
| RGO | 0.01% | 0.005% | 0.01% |

The viscosity x shear rate curves of the resin with and without graphene, have the same profile and are very close in terms of values, showing that graphene does not cause significant change in this property as can be seen from FIG. 8.

The addition of graphene or other carbon structure in the composition of the medical device of the present invention generates improvement in the mechanical properties of the device and it also makes the device more radiopaque, which helps in the visualization of the graft in imaging exams as can be verified from FIG. 13.

### D. The Tests Below Referred to the Embodiment of the Present Invention Comprising PDO Membrane

### Evaluation of the Bioactivity of the Medical Device of the Present Invention with PDO Membrane Using Cell-Based Tissue Engineering for Guided Bone Regeneration in Critical Calvary Defects of Rats

Methods: Male rats were divided into three groups: (1) medical device (β-tricalcium phosphate)+PDO membrane, (2) medical device comprising steem cells (β-tricalcium phosphate+adipose-derived stem cells (ASCs))+PDO membrane, and (3) medical device comprising steem cells (β-tricalcium phosphate+adipose-derived stem cells (ASCs))+PDO membrane comprising steem cells.

A surgical defect in the right parietal bone was made, and the defect was filled with the grafts mentioned above. The animals were euthanized 7, 14, and 30 days after the surgical procedure for histomorphometric and immunolabeling analyses.

Results: Cell-based therapy promotes, especially in group 3, a bone area formation at the defect border region and the center of the defect.

Conclusions the use of the combination of the medical device and the PDO membrane associated with cell-based therapy has excellent potential to improve and accelerate guided bone regeneration. Moreover, the use of ASCs optimized the bioceramic by increasing its osteoinductive and osteoprogenitor capacity even with the resorption of the printed scaffold.

At the end of the experiment, 30 days after the surgical procedure, the group 1 has the presence of bone tissue formation in the defect border region but still with a significant presence of connective tissue, which migrates inside the defect with the biomaterial widely resorbed. Group 2 presents the scaffold practically resorbed and filled by highly vascularized connective tissue in its interior. The defect border gives a large bone tissue formation, also migrating to the central region of the defect. Group 3 shows a large formation of bone tissue in the defect border area that migrates to the central region, with the presence of lacunae filled with osteocytes characterizing the organization and maturity of the bone. The presence of highly organized connective tissue, with pre-cursor cells for bone formation, extends to the central region, replacing the highly resorbed scaffold.

TABLE 3
Percentage of bone, connective tissue, and biomaterial
areas at 7, 14, and 30 days in group 1.

| | Bone | Connective Tissue | Biomaterial |
|---|---|---|---|
| 7 days | 1.940 | 29.453 | 25.044 |
| 14 days | 5.467 | 25.925 | 17.107 |
| 30 days | 8.460 | 23.281 | 19.047 |

Results in percentage (%) obtained from the histomorphometric analysis.

TABLE 4
Percentage of bone, connective tissue, and biomaterial
areas at 7, 14, and 30 days in group 2.

| | Bone | Connective Tissue | Biomaterial |
|---|---|---|---|
| 7 days | 4.585 | 28.571 | 26.984 |
| 14 days | 3.703 | 18.694 | 5.820 |
| 30 days | 9.347 | 52.380 | 8.289 |

Results in percentage (%) obtained from the histomorphometric analysis.

TABLE 5
Percentage of bone, connective tissue, and biomaterial
areas at 7, 14, and 30 days in group 3.

| | Bone | Connective Tissue | Biomaterial |
|---|---|---|---|
| 7 days | 5.996 | 27.336 | 20.105 |
| 14 days | 5.467 | 40.563 | 7.407 |
| 30 days | 16.754 | 32.451 | 10.758 |

Results in percentage (%) obtained from the histomorphometric analysis.

TABLE 6
Scores of TCP/PG (group 1), TCPasc/PG (group 2),
and TCPasc/PGasc (group 3) immunolabeling analysis.
Antibodies against IL-6, OPN, and OCN.

| | TCP/PG | TCPasc/PG | TCPasc/PGasc |
|---|---|---|---|
| IL-6 | +/+ | ++/++ | +/+ |
| OPN | +/++ | ++/++ | ++/++ |
| OCN | ++/++ | ++/++ | ++/++ |

Scores are evaluated at the border/center of the defect. Discrete labeling (+), moderate labeling (++), and intense labeling (+++).

### Preparation Process of the Present Invention

The process to prepare the present invention comprises, in summary, the following steps:

### A. Pacient-Specific Medical Device

a) Customer requests customized product, patient-specific model
b) Sending of imaging exams (.DICOM)
c) Treatment of DICOM
d) Planning and virtual modeling of the patient-specific bone graft
e) Approval of the product planned/modeled by the customer (physician or dentist)
f) Generated the .STL for printing

### B. Shape-Predefined Medical Device

a) Technical drawings of standard models (block, wedge and cylinder)
b) Archives. STL ready and already approved by the project team and technical responsible
c) Customization of the graft (device with fixed shape) and adaptation to the receiving bed:
   Customize the graft according to the anatomy of the receiving bed with the aid of drills and sterile surgical/prosthetic discs for straight pieces (examples: 702 drills, models of the Maxicut line and diamond blades);
   Give adequate finish, leaving no sharp edges and corners to avoid perforation of the flap;
   Make adaptation of the block to the receiving bed without leaving steps, leveling all sides of the graft to the bone defect.

Further, after the steps described above:
Primary and secondary assembly;
Gamma Irradiation Sterilization
Inspection;
Assembly of the outer packaging and
Labeling and Inventory.

### Brief Description of the Steps of the Preparation Process

The process for preparation of the present invention is illustrated in FIG. 11 and detailed below:

Virtual planning: The virtual planning of the models of the patient-specific medical devices is carried out by specialized technical team of projects for the modeling of the planning proposed and approved by the client. From the examination of images of the patient (example: computed tomography, DICOM file) employing CAD software is carried out the planning and modeling of the patient-specific product, once the planning is completed, the Planning Report is sent for approval of the client (dentist or physician). After approval, the file in STL format (in binary encoding) is imported by the print preparation software, where the parameters related to the raw material are added and sent to the printer via a wireless connection or cable.

An automatic dispenser is responsible for feeding the raw material (ceramic-based resin with β-TCP crystalline phase) into the tank during printing, preserving the necessary thickness for each layer to be photopolymerized (printed), without interventions during the printing process. In this way, the manufacturing process for both families is identical, via additive manufacturing employing Lithography-based Ceramic Manufacturing (LCM) technology and the same raw material.

Post processing: When printing is complete, parts are removed from the printing platform and cleaned with organic solvent to eliminate excess uncured resin from its surface. Subsequently, to ensure the cleanliness of the internal porous structure, the parts are submerged in solvent and subjected to a heated ultrasonic bath. After the external and internal cleaning step, the excess solvent is removed with a jet of compressed air then stored in a drying oven for 24 h. After the drying process, the employee positions the manufactured parts (identified by the production order, either special or standard) on alumina plates and selects the sintering program in an automated muffle furnace, with exhaust system and catalyst. The heat treatment (sintering) ensures that the burning of organic matter and the sintering of ceramic particles at 1200° C.

Having described an example of a preferred embodiment of the present invention, it should be understood that the scope of the present invention encompasses other possible variations of the inventive concept described, being limited only by the content of the appended claims, including possible equivalents therein.

1-19. (canceled)

20. A medical device capable of being used as a synthetic bone graft, said device being comprised of a synthetic bioceramic comprising a material selected from the group consisting of β-tricalcium phosphate and hydroxyapatite;
wherein said device is produced by additive manufacturing;
wherein said device is porous, and said porosity is planned and distributed according to a specific application; and
wherein said device is personalized or has a predefined-shape.

21. The medical device according to claim 20, wherein said device further comprises a carbon nanostructure.

22. The medical device according to claim 21, wherein the carbon nanostructure is selected from the group consisting of graphene, graphene oxide, reduced graphene oxide, and carbon nanotubes.

23. The medical device according to claim 22, wherein said device comprises graphene in a concentration that varies from 0.001% to 0.01%.

24. The medical device according to claim 20, wherein said device comprises:
a) a macropore having a diameter of from 360 μm to 440 μm;
b) a micropore having eexternal surface pores from 0.62 μm to 0.76 μm, and cross-sectional pores from 0.55 μm to 0.67 μm; and
c) a microstructure having a porosity of from 25.0% to 30.0%; an average diameter of from 0.63 to 0.77 μm; a penetration rate of from 0.023 cm³/g to 0.029 cm³/g, an average density of from 2.892 g/cm³ to 3.534 g/cm³, and 80% of pores: having a size of from 0.39 μm to 5.47 μm.

25. The medical device according to claim 20, wherein said device comprises stem cells:

26. The medical device according to claim 25, wherein the stem cells are autogenous adult mesenchymal stem cells obtained from a dermal punch of a patient.

27. The medical device according to claim 20, wherein the synthetic bioceramic comprises β-tricalcium phosphate (≥95% of β-TCP).

28. The medical device according to claim 20, wherein the synthetic bioceramic comprises hydroxyapatite (≥95% of HA).

29. The medical device according to claim 20, wherein said device is a patient-specific medical device planned and built virtually based on data acquired by computed tomography or magnetic resonance using virtual 3D models and CAD/CAM techniques.

30. The medical device according to claim 20, wherein said device is a predefined-shape medical device that is customized by a surgeon.

31. The medical device according to claim 20, wherein said device comprises internal filling.

32. The medical device according to claim 31, wherein said internal filling has a gyroid shape.

33. The medical device according to claim 20, wherein said device comprises a polydioxanone polymer membrane.

34. A process for preparing the medical device according to claim 20, comprising:
a. examining a patient's images obtained using CAD software;
b. generating a planning report file in STL format in binary encoding which is sent for approval by a dental surgeon;
c. upon obtaining approval of the planning report file, said file is imported by print preparation software, wherein parameters relating to raw material are added, and said file is sent to a printer via a wireless or cable connection; and
d. said medical device is produced by additive manufacturing based on said planning report file.

35. The process according to claim 34, wherein the patient's images are obtained from computed tomography or a DICOM file.

36. A process for preparing the medical device according to claim 20, comprising:
a. preparing technical drawings of standard models;
b. preparing an archive of said technical drawings when approved by a technical project team;
c. customizing a graft according to the anatomy of a receiving bed with the aid of drills and sterile surgical/prosthetic discs for straight pieces;
d finishing said device so as to leave no sharp edges and corners to avoid perforation of a flap on the device; and
e. adapting a block to the receiving bed without leaving steps, and leveling all sides said device to prepare a graft for a bone defect.

37. The process according to claim 36, wherein the standard models are a block, a wedge, or a cylinder.

38. A process for insertion of the medical device according to claim 20 comprising:
a. conducting an initial site assessment to check for clinical defects;
b. detaching tissue to expose a surgical bed;
c. using a polymeric guide to verify insertion of said medical device;
d perforating the surgical bed to allow blood perfusion and nutrition of said medical device; and
e. adapting said medical device on the surgical bed via screw stabilization.

39. The process according to claim 38 wherein the polymeric guide is a bone graft analog produced by additive manufacturing.

40. A method of using the medical device according to claim 20 comprising using said medical device to function as a bone graft both for volume augmentation/reconstruction of cranio-maxillofacial, neurocranium, long bones and spine defects, and to provide maintenance space wherein said medical device is gradually replaced by newly formed bone.

41. The method according to claim 40 wherein said medical device is used for a process selected from the group consisting of bone reconstructions for horizontal and/or vertical augmentation, filling of intraosseous defects; traumatology and bone reconstructions for horizontal and/or vertical augmentation; and filling of intraosseous bone defects.
